SEQUENCE LISTING (2 sequences)

SEQ ID NO: 1 - DNA, 475 bp, Artificial
  nucleic acid sequence encoding the recombinant human IL-1 receptor antagonist Anakinra
catatgcgaccgtccggccgtaagagctccaaaatgcaggctttccgtatctgggacgttaaccagaaaaccttctacctgcgcaacaaccagctggttgctggctacctgcagggtccgaacgttaacctggaagaaaaaatcgacgttgtaccgatcgaaccgcacgctctgttcctgggtatccacggtggtaaaatgtgcctgagctgcgtgaaatctggtgacgaaactcgtctgcagctggaagcagttaacatcactgacctgagcgaaaaccgcaaacaggacaaacgtttcgcattcatccgctctgacagcggcccgaccaccagcttcgaatctgctgcttgcccgggttggttcctgtgcactgctatggaagctgaccagccggtaagcctgaccaacatgccggacgaaggcgtgatggtaaccaaattctacttccaggaagacgaataatgggaagctt

SEQ ID NO: 2 - protein, 153 aa, Artificial
  nucleic acid sequence encoding the recombinant human IL-1 receptor antagonist Anakinra
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

(19) United States
(12) Patent Application Publication
(10) Pub. No.: US 2010/0310575 A1
(43) Pub. Date: Dec. 9, 2010

(54) Inhibitors of Caspase I-Dependent Cytokines in the Treatment of Neurodegenerative Disorders

(75) Inventors: Arturo Zychlinsky, Berlin (DE); Felix Meissner, Berlin (DE); Kaaweh Molawi, Berlin (DE); Thomas Meyer, Berlin (DE)

Correspondence Address: FULBRIGHT & JAWORSKI L.L.P., 600 CONGRESS AVE., SUITE 2400, AUSTIN, TX, 78701, US

(73) Assignee: MAX-PLANCK-GESELLSCHAFT ZUR FORDERUNG DER WISSENSC

(21) Appl. No.: 12/739,118

(22) PCT Filed: Oct. 24, 2008
(86) PCT No.: PCT/EP08/09039

§ 371 Date: Aug. 26, 2010

Related U.S. Application Data
(60) Provisional application No. 60/996,058, filed on Oct. 26, 2007.

(51) Int. Cl. A61K 39/395; C07K 16/24; C07H 21/04; A61K 31/7088; C07H 21/02; A61K 31/7105; A61K 38/16; C07K 14/00; A61P 25/28; A61P 25/16; A61P 25/02

(52) U.S. Cl. 424/158.1; 530/389.2; 536/24.5; 514/44 A; 536/23.1; 514/44 R; 514/17.7; 530/350; 514/18.2; 514/17.8

(57) ABSTRACT

The present invention relates to a method for treating, preventing or ameliorating a chronic neurodegenerative disorder, in particular progressive muscular atrophy (PMA), said method comprising administering to a subject in need of such a treatment, prevention or amelioration a specific inhibitor of a caspase I-dependent cytokine. Also specific inhibitors of a caspase I-dependent cytokine for treating, preventing or ameliorating a neurodegenerative disorder, in particular PMA, are disclosed herein. Furthermore, the present invention provides for the use of (a) specific inhibitor(s) of a caspase I-dependent cytokine in the medical or pharmaceutical intervention of neurodegenerative disorders. In particular said cytokine to be inhibited is selected from the group consisting of interleukin-1 (IL-1), interleukin-18 (IL-18), interleukin-33 and interferon γ (IFN-γ; interferon-gamma) and most preferably said inhibitor to be employed in context of this invention is a human interleukin-1 receptor antagonist (IL-1Ra), like anakinra.

The present invention relates to a method for treating, preventing or ameliorating a chronic neurodegenerative disorder, in particular progressive muscular atrophy (PMA), said method comprising administering to a subject in need of such a treatment, prevention or amelioration a specific inhibitor of a caspase I-dependent cytokine. Also specific inhibitors of a caspase I-dependent cytokine for treating, preventing or ameliorating a neurodegenerative disorder, in particular (PMA), are disclosed herein. Furthermore, the present invention provides for the use of (a) specific inhibitor(s) of a caspase I-dependent cytokine in the medical or pharmaceutical intervention of neurodegenerative disorders. In particular said cytokine to be inhibited is selected from the group consisting of interleukin-1 (IL-1), interleukin-18 (IL-18), interleukin-33 and interferon γ (IFN-γ; interferon-gamma) and most preferably said inhibitor to be employed in context of this invention is a human interleukin-1 receptor antagonist (IL-1 Ra), like anakinra.

Several documents are cited throughout text of this specification. Each of the documents cited herein (including any manufacturer's specifications, instructions, etc.) are hereby incorporated by reference.

Neurodegenerative disorders are often chronic conditions, like lower motor neuron disease, in particular progressive muscular atrophy (PMA), amyotrophic lateral sclerosis (ALS), Alzheimer's disease (AD), Parkinson's disease or Huntington's disease. Yet, neurodegenerative disorders also comprise acute conditions, like ischemia and head or spinal cord injuries. In both, chronic as well as acute conditions, inflammatory processes and corresponding inflammatory factors (like cytokines) have been proposed. Furthermore, certain autoimmune disorders were related to an inflammatory component, like myasthenia gravis or multiple sclerosis. Whereas the mechanisms for neuroinflammation, in particular in the Case of neuronal cells, have been studied in several in vitro models, the role of cytokines in neuronal disorders and in particular in chronic neurodegeneration is still not understood.

In Alzheimer's disease, the inflammatory component was recognized and since the early 1990ties, it was proposed to use anti-inflammatory drugs, like non-steroidal anti-inflammatory drugs (NSAIDS), in the prevention or amelioration of dementia. Alzheimer's disease is characterized by neurofibrillary tangles in particular in pyramidal neurons of the hippocampus and numerous amyloid plaques containing mostly a dense core of amyloid deposits and defused halos. The extracellular neuritic plaques contain large amounts of a pre-dominantly fibrillar peptide termed “amyloid β”, “A-beta”, “Aβ4”, “β-A4” or “Aβ”. This amyloid β is derived from “Alzheimer precursor protein/β-amyloid precursor protein” (APP). It is known in the art that cholinergic agonists and interleukin 1 regulate processing and secretion of the Alzheimer beta/A4 amyloid protein precursor (see, Buxbaum (1992), PNAS 89, 10075) and that there is a reciprocal control of IL-1 as well as IL-6 and beta-amyloid production in cultures (see, Del Bo (1995), Neuroscience Letters 188, 70). Furthermore, it was reported that A-beta may stimulate the release of inflammatory cytokines, like IFN-gamma and IL-1 (Lindberg (2005) J. Mol. Neuroscience 27, 1).

Amyotrophic lateral sclerosis (ALS) is a neurodegenerative disease that causes progressive paralysis of affected patients. ALS is hallmarked by upper and lower motor neuron damage which results in loss of motor control and the degeneration of the denervated muscles. Other symptoms may include difficulties in speaking, breathing and swallowing, spasticity, muscle cramps and weakness. ALS is one of the most common neuromuscular diseases with an incidence of 1 to 2 new cases per 100,000 people per year. Although death typically occurs within 5 years of the initial diagnosis, about 10% of patients diagnosed with ALS survive for 10 or more years. Different types of ALS have been identified including a familial and a sporadic variant whereby familial ALS accounts for up to 10% of all cases. The affected neurons die of apoptosis by a mechanism that is not understood. Yet, it has been suggested that environmental factors such as viral infection, exposure to neurotoxins or heavy metals or genetic factors such as enzyme abnormalities may play a role. Particularly, some forms of familial ALS have been linked to mutations in the superoxide dismutase enzyme SOD1; see Cleveland (2001) Nat. Rev Neurobiol. 2: 806. SODs are a ubiquitous family of enzymes that convert superoxide anions to water and hydrogen peroxide; see Fridovich (1975) Annu Rev Biochem 44:147-159. Many mutations in SOD1 lead to ALS (Rosen (1993) Nature 364:362) and different mutants (mtSOD1) have various levels of enzyme activities, indicating that the phenotype is not due to a loss of function; see Gurney (1994) Science 264:1772-1775 and Wong (1995) Neuron 14:1105-1116. Mice expressing a transgenic human mtSOD1, but not mice expressing wild type SOD nor SOD1 deficient animals, develop a hind-limp paralysis and die at an early age, reminiscent of ALS; see Gurney (1994) loc.cit. and Wong (1995) loc.cit. However, the etiologic link between SOD1 mutations and neuronal death is not yet understood. Mice expressing transgenic human mtSOD1 presently represent the best animal model for ALS. There is no effective treatment for ALS. Currently, Riluzole is the only FDA approved medicament for the treatment of ALS. Riluzole is thought to reduce the glutamate signalling by antagonizing the NMDA receptor and by blocking sodium channels associated with damaged neurons; see Song (1997) J. Pharmacol Exp Ther 282:707-714. Although riluzole may slow down the progression of the disease no subjective improvement of the patient's condition could be reported. Furthermore, riluzole treatment is associated with side effects including liver toxicity, neutropenia, nausea and fatigue (Wagner (1997) Ann Pharmacother 31:738-744).

Some investigators have speculated that caspase 1 and caspase 3 may have a functional role in ALS. Therefore, Zhu (2002) in Nature 417, 74 has proposed that minocycline inhibits cytochrome c release and delays progression of ALS in a mouse model, whereby minocycline is a inhibitor of caspase-I and caspase-3 transcription. Similarly, Friedlander and colleagues (1997) have speculated that interleukin-1 beta-converting enzyme (ICE, caspase I) like proteases may affect disease progression in a particular mouse model of ALS; see Nature 388, page 31. In Li (2000) Science 288, 335 it is also suggested that the general inhibition of caspase may have a protective effect in ALS.

Kim (2006) annals of Neurology 60, 716 proposes a correction of humoral derangements, namely an increase of IL-1 beta, Il-6, IL-12 and vascular endothelial growth factor (VEGF), from mutant SOD 1 in spinal cord by a “cocktail approach” employing a combination of neutralizing antibodies to all these cytokines. It is taught that only and merely a combinatorial approach to the treatment of inflammation in ALS might protect motor neurons.

Yet, as is also stated in Schultzberg (2007), Phys. Behav. 92, 121, the role of cytokines in the nervous system, particularly the possibility to use anti-inflammatory or anti-cytokine measures for neuroprotective purposes is far from elucidated. Furthermore, there are serious down-sites to use broad inhibitors of the cytokine system and pathways since inhibition of e.g. caspases lead to serious and undesired side effects.

Accordingly, the technical problem underlying the present invention is the provision of means and methods that improve the medical situation of human patients suffering from a chronic neurodegenerative disorder, like lower motor neuron disease, in particular progressive muscular atrophy (PMA), ALS, Alzheimer's disease, Parkinson's disease, or Huntington's disease.

The technical problem is solved by the embodiments as characterized in the claims and as described herein below.

Accordingly, the present invention relates to a method for treating, preventing or ameliorating a chronic neurodegenerative disorder said method comprising administering to a subject in need of such a treatment, prevention or amelioration a specific inhibitor of a caspase I-dependent cytokine. Also provided is a specific inhibitor of a caspase I-dependent cytokine for treating, preventing or ameliorating a neurodegenerative disorder and the invention, in a further embodiment, relates to the use of specific inhibitor of a caspase I-dependent cytokine in the medical and pharmaceutical intervention of chronic neurodegenerative disorders, like lower motor neuron disease, in particular PMA, ALS, Alzheimer's disease, Parkinson's disease, or Huntington's disease. In a particular preferred embodiment, the chronic neurodegenerative disorder to be treated is PMA and the specific inhibitor of a caspase I-dependent cytokine to be employed is an inhibitor of interleukin-1 (IL-1).

Yet, also within gist of the present invention is the medical sue of further inhibitors of caspase I-dependent cytokines, whereby said cytokines to be specifically inhibited are selected from the group consisting of, interleukin-1 beta, (IL-1 beta), Interleukin-1 alpha (IL-1 alpha), interleukin-18 (IL-18), interleukin-33 (IL-33) and interferon gamma (IFN-gamma). However, as is illustrated in the appended scientific data and corresponding figures, in a most preferred embodiment, the chronic neurodegenerative disorder, in particular amyotrophic lateral sclerosis (ALS), is to be treated with a specific inhibitor of 1′-1 (alpha and/or beta), like anakinra (Kinerert®).

In accordance with the present invention and as documented in the appended figures and scientific results, it was surprisingly found that (a) individual and specific inhibitor(s) of individual caspase-I dependent cytokines can successfully be employed in the amelioration and thereby treatment of chronic neurodegenerative disorders. As shown herein, a specific and individual inhibitor of interleukin 1 (IL-1 alpha and/or beta), namely the peptide inhibitor anakinra (Kineret®) can be used in the treatment of amyotrophic lateral sclerosis (ALS). This finding is in stark contrast to the teachings in the prior art where it was proposed that chronic neurodegenerative disorders, in particular ALS, can only be tackled by the sue of broad inhibitors which target early enzymes in the caspase pathways. For example, it was taught that broad inhibitors of caspase-1 and caspase-3 (like minocycline that does not directly inhibit even these early enzymes) be used for mediation of neuroprotection in neurodegeneration; see, Zhu (2002, loc.cit.). Furthermore, it is of note that minocycline inhibits not only caspase-1 and caspase-3 but also the inducible form of nitric oxide synthetase and p38 mitogen-activated protein kinase (MAPK); see also Zhu (2002, loc. Cit). Similarly, Friedlander (1997, loc. Cit) had proposed that ICE inhibitors (i.e. inhibitors of caspase-1) may be of value in the treatment of ALS. Since caspases play a role in neurodegeneration in transgenic mouse models of ALS (SOD.mice; transgenic SOD1 G93A mice), Li and colleagues (2000) also suggest that broad caspase inhibition may have a protective role in ALS. More importantly, even recently, Kim and colleagues (2006; loc.cit) have employed these SOD mice and have taught that only a combinatorial approach of antibody inhibitors of Il-6, IL-12 and VEGF can be used in the treatment of ALS. Accordingly, and in contrast to the prior art, the present invention provides for the first time evidence and proof that the individual inhibition of single, late enzymes in the caspase-1 pathway can be used for a successful amelioration and treatment of chronic neurodegenerative disorders, and in particular of ALS.

As shown herein, in a specific embodiment of the present invention, the IL-1 receptor antagonist (IL1-RN; Il-1 Ra; IL1-Li) “Kineret®”/anakinra is employed in the treatment, prevention and/or amelioration of the neurodegenerative disorder ALS. In a specific embodiment, said ALS to be treated is the familial form of ALS (FALS), often correlated with a SOD mutation.

“Kineret®”/anakinra is well known in the art and is FDA-approved as a safe drug in the treatment of rheumatoid arthritis. “Kineret®”/anakinra is a recombinant human IL-1 receptor antagonist (see, e.g. Bresniham (1998), Art Rheum 43, 1001 or Campion (1996), Art. Rheum 39, 1092) and differs form the native human IL-1Ra in that it has the addition of a single methionine residue at its amino terminus. It consists of 153 amino acids and has a molecular weight of 17.3 kD. It is clear for the person skilled in the art that the present invention is not limited to the use of the marketed “Kineret ®”/anakinra but that also further specific inhibitors of caspase 1-dependent cytokines, in particular Il-1 (receptor) antagonists may be employed. For example, also homologous peptides to “Kineret®”/anakinra may be employed which comprise in certain positions of its amino acid sequence conservative or non-conservative replacements and/or exchanges. Also further functional derivatives, biological equivalents and functional mutations of the concrete “Kineret®”/anakinra peptide/protein may be employed in context of this invention as long as these compounds are capable of inhibition the biological function of 1′-1. Accordingly, the present invention is not limited to the medical and pharmaceutical use of “Kineret ®”/anakinra but also biological equivalents thereof. “Kineret®”/anakinra as well as biological equivalents, i.e. further IL-1 inhibitor proteins are known in art and, inter alia, described in WO89/11540, WO92/16221, WO95/34326, U.S. Pat. No. 5,075,222 (B1) or U.S. Pat. No. 6,599,873 (B1). The IL-1 inhibitors/antagonists to be used in context of this invention, for example and in one particular equivalent in the medical intervention of ALS is an IL-1 inhibitor that is a monocyte-derived IL-1 inhibitor (like “Kineret ®”/anakinra and its biological equivalents, functional mutations and derivatives). The person skilled in the art is readily in a position to prepare such inhibitors, as inter alia, illustrated in U.S. Pat. No. 6,599,873 (B1), U.S. Pat. No. 5,075,222 (B1), WO89/11540, WO92/16221 or WO 93/21946, WO94/06457, WO 95/34326, whereby in particular WO92/16221 and WO95/34326 provide for modified biological equivalents to IL1-receptors, like, e.g. pegylated version of soluble IL1-receptor antagonists. In U.S. Pat. No. 6,599,873 (B1), U.S. Pat. No. 5,075,222 (B1), WO89/11540, WO92/16221 or WO95/34326 preferred ways of production by recombinant methods are also provided.

The coding sequence as well as the amino acid sequence of the preferred Il-1 antagonist/inhibitor, i.e. of anakinra is known in the art and available under accession number CS182221 (gene sequence) in NCBI gene bank.

A corresponding coding sequence is provided here as SEQ ID NO. 1:

| | |
|---|---|
| | (SEQ ID No. 1) |
| 1 | catatgcgac cgtccggccg taagagctcc aaaatgcagg |
| | ctttccgtat ctgggacgtt |
| 61 | aaccagaaaa ccttctacct gcgcaacaac cagctggttg |
| | ctggctacct gcagggtccg |
| 121 | aacgttaacc tggaagaaaa aatcgacgtt gtaccgatcg |
| | aaccgcacgc tctgttcctg |
| 181 | ggtatccacg gtggtaaaat gtgcctgagc tgcgtgaaat |
| | ctggtgacga aactcgtctg |
| 241 | cagctggaag cagttaacat cactgacctg agcgaaaacc |
| | gcaaacagga caaacgtttc |
| 301 | gcattcatcc gctctgacag cggcccgacc accagcttcg |
| | aatctgctgc ttgcccgggt |
| 361 | tggttcctgt gcactgctat ggaagctgac cagccggtaa |
| | gcctgaccaa catgccggac |
| 421 | gaaggcgtga tggtaaccaa attctacttc caggaagacg |
| | aataatggga agctt |

and one amino aid sequence representing an IL-1 antagonist to be employed in accordance with this invention is represented in the following SEQ ID. NO. 2 in the one letter code:

(SEQ ID No. 2)
MRPSGRKSSKMQAFRIWDVNQKTFYLRNNQLVAGYLQGPNVNLEEKIDVV
PIEPHALFLGIHGGKMCLSCVKSGDETRLQLEAVNITDLSENRKQDKRFA
FIRSDSGPTTSFESAACPGWFLCTAMEADQPVSLTNMPDEGVMVTKFYFQ
EDE

Accordingly, the present invention also relates to modified versions and biological equivalents of IL-1 (receptor) antagonists, like “Kineret®”/anakinra. The appended scientific data provide for examples how the person skilled in the art can test whether such a “Kineret®”/anakinra mutation, biological equivalent or derivative is still functional.

Il-1 Inhibition test are known in the art and comprise, inter alia NF-kB reporter gene assays (Zhang et al. J Biol Chem vol. 279 2004)

Also other specific inhibitor of a caspase I-dependent cytokine are well known in the art and may comprise, but are not limited to compounds that are selected from the group consisting of neutralizing antibody or an antibody derivative or a fragment thereof to interleukin-1 (IL-1), interleukin-18 (IL-18), interleukin-33 (IL-33) and/or interferon gamma (IFN-gamma), antisense oligonucleotides specifically interacting with nucleic acid molecules encoding interleukin-1 (IL-1), interleukin-18 (IL-18) or interferon gamma (IFN-gamma), siRNA or RNAi directed against interleukin-1 (IL-1), interleukin-33 (IL-33), interleukin-18 (IL-18) or interferon gamma (IFN-gamma), ribozymes specifically interacting with nucleic acid molecules encoding for functional interleukin-1 (IL-1), interleukin-33 (IL-33), interleukin-18 (IL-18) or interferon gamma (IFN-gamma), an interleukin-1 (IL-1) antagonist, a interleukin-18 (IL-18) antagonist and a interferon gamma (IFN-gamma) antagonist. The antagonists of IL-1, IL-18 or IFN-gamma may, in particular be (a) corresponding receptor antagonist(s).

The term “inhibitor” or “antagonist” of a caspase 1-dependent cytokine (like IL-1, IL-33, IL-33 or IFN-gamma, and in particular 1′-1) is known in the art and easily understood by the skilled artisan. In accordance with the present invention, the term “inhibitor”/“antagonist” denotes molecules or substances or compounds or compositions or agents or any combination thereof described herein below, which are capable of inhibiting and/or reducing the natural cytokine action described herein and more particularly the receptor-mediated activity of IL-1. The term “inhibitor” when used in the present application is interchangeable with the term “antagonist”. The term “inhibitor” comprises competitive, non-competitive, functional and chemical antagonists as described, inter alia, in Mutschler, “Arzneimittelwirkungen” (1986), Wissenschaftliche Verlagsgesellschaft mbH, Stuttgart, Germany. The term “partial inhibitor” in accordance with the present invention means a molecule or substance or compound or composition or agent or any combination thereof that is capable of incompletely blocking the action of agonists through, inter alia, a non-competitive mechanism. It is preferred that said inhibitor alters, interacts, modulates and/or prevents either the biosynthesis of the caspase1-dependent cytokine (in particular IL-1) in a way which leads to partial, preferably complete, standstill or it alters, interacts, modulates and/or prevents the biological function of said cytokine. Said standstill may either be reversible or irreversible. The inhibitors to be employed in accordance with this invention may by biological inhibitors (like, e.g. “Kineret®”/anakinra for the inhibition of IL-1); the Il-18 binding molecule (IL-18 by or Tadakinig-alpha; see, e.g. WO 99/09063) for the inhibition of IL-18; the soluble IFN-gamma receptor (as, inter alia described in Michiels (1998), J. Biochem Cell Biol. 30, 505) for the inhibition of IFN-N, or may also be a chemical inhibitor, like a small molecule.

The person skilled in the art can easily employ the compounds and the methods of this invention in order to elucidate the inhibitory effects and/or characteristics of a test compound to be identified and/or characterized in accordance with any of the methods described herein and which is an inhibitor of a caspase-1 dependent cytokine, like in particular of IL-1.

The term “test compound” or “compound to be tested” refers to a molecule or substance or compound or composition or agent or any combination thereof to be tested by one or more screening method(s) of the invention as a putative inhibitor of an inhibitor of a caspase-1 dependent cytokine, like in particular of IL-1. A test compound can be any chemical, such as an inorganic chemical, an organic chemical, a protein, a peptide, a carbohydrate, a lipid, or a combination thereof or any of the compounds, compositions or agents described herein. It is to be understood that the term “test compound” when used in the context of the present invention is interchangeable with the terms “test molecule”, “test substance”, “potential candidate”, “candidate” or the terms mentioned hereinabove.

Accordingly, small peptides or peptide-like molecules as described herein below are envisaged to be used in the screening methods for inhibitor(s) of an inhibitor of a caspase-1 dependent cytokine, like in particular of IL-1. Such small peptides or peptide-like molecules bind to and occupy the active site of a protein thereby making the catalytic site inaccessible to substrate such that normal biological activity is prevented. Moreover, any biological or chemical composition(s) or substance(s) may be envisaged as an inhibitor of a caspase-1 dependent cytokine, like in particular of IL-1. The inhibitory function of the inhibitor can be measured by methods known in the art and by methods described herein. Such methods comprise interaction assays, like immunoprecipitation assays, ELISAs, RIAs as well as specific inhibition assays, like the assays provided in the appended examples (e.g. enzymatic in vitro assays) and inhibition assays for gene expression. In the context of the present application it is envisaged that cells expressing an inhibitor of a caspase-1 dependent cytokine, like in particular of IL-1. Such cells are e.g. peritoneal macrophages capable of expression e.g. IL-1. Cells expressing the receptors for caspase-1 dependent cytokine, like IL-1 receptor, comprise e.g natural killer cells, macrophages, neutrophils, and the like.

Further know test system for the functionality of an inhibitor of a caspase-1 dependent cytokine comprise, e.g. ELISA analysis of cytokine release after activation of macrophages and neutrophils and induction of shock in mice.

Also preferred potential candidate molecules or candidate mixtures of molecules to be used when contacting an element of the IL-1 and IL-1 receptor interaction, may be, inter alia, substances, compounds or compositions which are of chemical or biological origin, which are naturally occurring and/or which are synthetically, recombinantly and/or chemically produced.

Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means.

In addition, the generation of chemical libraries is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds can theoretically be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop, Journal of Medicinal Chemistry, Vol. 37, No. 9, 1233-1250 (1994)). Other chemical libraries known to those in the art may also be used, including natural product libraries. Once generated, combinatorial libraries are screened for compounds that possess desirable biological properties. For example, compounds which may be useful as drugs or to develop drugs would likely have the ability to bind to the target protein identified, expressed and purified as described herein.

In the context of the present invention, libraries of compounds are screened to identify compounds that function as inhibitors of the target gene product, here caspase-1 dependent cytokine, like in particular of IL-1. First, a library of small molecules is generated using methods of combinatorial library formation well known in the art. U.S. Pat. Nos. 5,463,564 and 5,574,656 are two such teachings. Then the library compounds are screened to identify those compounds that possess desired structural and functional properties. U.S. Pat. No. 5,684,711, discusses a method for screening libraries. To illustrate the screening process, the target cell or gene product and chemical compounds of the library are combined and permitted to interact with one another. A labelled substrate is added to the incubation. The label on the substrate is such that a detectable signal is emitted from metabolized substrate molecules. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the cell/enzyme can be analyzed and features common to the various compounds identified can be isolated and combined into future iterations of libraries. Once a library of compounds is screened, subsequent libraries are generated using those chemical building blocks that possess the features shown in the first round of screen to have activity against the target cell/enzyme. Using this method, subsequent iterations of candidate compounds will possess more and more of those structural and functional features required to inhibit the function of the target cell/enzyme, until a group of inhibitors with high specificity for the enzyme can be found. These compounds can then be further tested for their safety and efficacy as antibiotics for use in animals, such as mammals. It will be readily appreciated that this particular screening methodology is exemplary only. Other methods are well known to those skilled in the art. For example, a wide variety of screening techniques are known for a large number of naturally-occurring targets when the biochemical function of the target protein is known.

Preferably, candidate agents encompass numerous chemical classes, though typically they are organic molecules, preferably small organic compounds having a molecular weight of more than 50 and less than about 2,500 Daltons, preferably less than about 750, more preferably less than about 350 daltons.

Candidate agents may also comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or poly-aromatic structures substituted with one or more of the above functional groups.

Exemplary classes of candidate agents may include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

As mentioned above, candidate agents are also found among biomolecules including peptides, amino acids, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Other candidate compounds to be used as a starting point for the screening of inhibitors of the biosynthesis or biological function of caspase-1 dependent cytokines, like in particular of IL-1, are aptamers, aptazymes, RNAi, shRNA, RNAzymes, ribozymes, antisense DNA, antisense oligonucleotides, antisense RNA, neutralizing antibodies, affybodies, trinectins or anticalins.

The person skilled in the art knows already about useful inhibitors of caspase-1 dependent cytokine, like of IL-1, IL-33, IL-18 and/or IFN-gamma. For example, an known interleukin-18 (IL-18) antagonist is interleukin-18 binding protein (Tadakinig-alpha). A known inhibitor/antagonist of interferon gamma (IFN-gamma) may be soluble IFN-gamma receptor (as, inter alia described in Michiels (1998), J. Biocehm Cell Biol. 30, 505)

Also interleukin-1 (IL-1) antagonists/inhibitors are known in the art and comprise, e.g. IL-1 TRAP, (Economides et al., nat med 2003), CDP 484 (Braddock et al. Nat Rev drug discovery 3, 2004), the soluble IL-1 receptor accessory protein (sIL-1 RAcP; Smeets et al. Arthritis rheum 48, 2003) and the decoy receptor IL-1RII (Neumann et al. J Immunol 165, 2000). The most preferred IL-1 antagonist/inhibitor in context of this invention is “Kineret®”/anakinra, in particular in the treatment of a lower motor neuron disease (such as progressive muscular atrophy or spinal muscular atrophy) or ALS.

However, it also envisaged in context of this invention that the herein described inhibitors/antagonists of caspase-1 dependent cytokines, like in particular of IL-1 are also employed in the treatment of chronic neurodegenerative disorder like Huntington's disease, Alzheimer's disease, or Parkinson's disease.

The term “lower motor neuron disease” as used herein refers to diseases wherein the lower motor neurons are clinically affected (e.g. degenerated or damaged). In sporadic forms of LMND, such as progressive muscular atrophy (PMA), the above-mentioned degeneration or destruction of lower motor neurons causes symptoms that vary from primarily bulbar signs to distal or proximal limb involvement. Also the treatment of hereditary forms of LMND, like spinal muscular atrophy (SMA) or Kennedy's syndrome is envisaged in context of the present invention. Accordingly, in a particularly preferred embodiment of the present invention, lower motor neuron diseases, such as progressive muscular atrophy (PMA), spinal muscular atrophy (SMA) and Kennedy's disease are to be treated, prevented and/or ameliorated.

The term “progressive muscular atrophy (PMA)” refers in context of the present invention to a progressive neurological disease in which exclusively the lower motor neurons deteriorate causing atrophy and fasciculation. In contrast to ALS, PMA is not rapidly progressive. In ALS, all motor neurons can be affected and progression can be either slow or fast. Again, in contrast to ALS, in PMA upper motor neuron difficulties such as spasticity, brisk reflexes, or the Babinski sign are absent. Furthermore, in PMA inclusions, such as Lewy-body like hyaline inclusions or Bunina-bodies (Matsumoto, Clin Neuropathol. 1996 15(1), 41-6) are exclusively found in lower motor neurons, whereas in ALS these inclusions also occur in the brain stem. These inclusions are preferably detected by standard histological assays like H and E stainings and immunohistochemistry. Patients suffering from ALS and PMA also differ in the average survival rate. The typical survival rate for ALS is approximately 2 to 5 years after initial diagnosis. In PMA survival is in the order of 5-10 years. PMA patients do also not suffer from the cognitive changes that can affect ALS patients.

In the art, it is believed that two PMA subtypes exist, one with a patchy distribution and one with a leg distribution. In the first case, progression is unpredictable, whilst in the latter there is a prolonged latency period between the progression from legs to arms, and then again to the bulbar region.

It is known that patients suffering from PMA survive longer than ALS patients. In some cases symptoms in PMA patients can be restricted to the arms or legs for a long period of time before spreading elsewhere in the body. In the present invention, it has been surprisingly found that PMA patients can be effectively treated with a specific inhibitor of a caspase I-dependent cytokine, in particular with IL-1R^{N} (Anakinra/Kineret®). In context of this invention, PMA patients show a better response effect to IL-1RN compared to ALS patients. Without being bound by theory, the better response in PMA patients may be based on the fact that PMA patients show a slower progression of the disease than ALS patients.

As a further advantage, IL-1RN is preferably injected peripherally, thus being more accessible to the neurons affected in PMA than in ALS. Peripheral injection of IL-1RN does, therefore, also increase the response to IL-1RN in PMA patients. In ALS, many of the affected neurons are beyond the blood brain barrier and are therefore not accessible to IL-1RN.

As mentioned above, also hereditary forms of LMND can be treated, prevented and/or ameliorated with a specific inhibitor of a caspase I-dependent cytokine in accordance with the present invention. Exemplary hereditary forms of LMND such as spinal muscular atrophy (SMA) and Kennedy's syndrome are described below.

Spinal muscular atrophy (SMA) is a genetic disease that affects muscle movement. It causes the motor of the anterior horn to deteriorate. The loss of lower motor neuron activity causes weakening and atrophy of the muscles. SMA is classified into four types, based on the age at which it develops and the severity of the symptoms. Types I, II and III develop in childhood. Type IV is SMA that starts in adulthood, with symptoms usually beginning over the age of 35.

SMA Type I—also known as Werdnig-Hoffman disease, SMA Type I is the most severe form of the disease. It can develop from before birth (some mothers notice decreased movement of the fetus in the final months of their pregnancy), up to six months of age. The patients are never able to sit and die of respiratory insufficiency before the age of 2 years. SMA Type II—this is the intermediate form of the disease and develops between 6-18 months of age. The patients are never able to stand, life expectancy is shortened. SMA Type III—also known as Kugelberg-Welander disease, is the least severe childhood form of the disease. It develops between 18 months-17 years of age. Functional losses appear gradually and vary significantly. Life expectancy can be normal. SMA Type IV—this is SMA that begins in adulthood and is usually a milder form of the disease than Types I, II and III. There is also an adult form of SMA—called Kennedy's syndrome or spinal-bulbar muscular atrophy—that occurs only in men. Kennedy's syndrome usually develops between 20-40 years of age, although it can affect men from their teens to their 70s.

SMA is thought of being caused by a defective gene. The childhood SMAs (Types I, II and III) are all autosomal recessive diseases. About 1 in 40 people carry the defective gene. SMA that begins in childhood is rare, affecting 4 children in every 100,000. SMA that begins in adulthood is even less common, affecting about 1 person in every 300,000. SMA can affect both males and females, although it is more common in males, particularly in those who develop the disease between 37 months and 18 years of age. SMA types I-III have been mapped to chromosome 5q11.2-13.3 four genes: the SMN gene, the NAIP gene, the p44 and the H4F5 gene.

Kennedy's Syndrome is an X-linked inherited late onset proximal spinal and bulbar muscular atrophy with slow progression. It is believed that this disease is caused by an expansion of CAG trinucleotide repeats in the first exon of the androgen receptor gene resulting in proteins with polyglutamin expansions that tend to aggregate (similar to Huntington's disease). Many of these aggregates are ubiquitin positive. As mentioned above, also Huntington's disease can be treated in accordance with the present invention with a specific inhibitor of a caspase I-dependent cytokine, in particular IL-1RN.

A preferred medical intervention as described in the present invention is the treatment, prevention and/or amelioration of amyotrophic lateral sclerosis (ALS) and in particular familial amyotrophic lateral sclerosis (FALS). Said familial amyotrophic lateral sclerosis (FALS) may be linked to a mutation/variant of the Cu/Zn superoxide dismutase (SOD1).

It is understood that the patients to be treated with an specific inhibitor/antagonist of a caspase1-dependent cytokine, in particular an inhibitor of IL-1, in context of the present invention is a human patient. Yet, this invention is not limited to the medical intervention on human beings.

Furthermore, it is within the scope of the present invention that the specific inhibitor/antagonist of a caspase1-dependent cytokine, in particular the inhibitor of IL-1 as described herein, may be employed in co-therapy approaches. For example, it is envisaged that “Kineret®”/anakinra, as an example of a specific IL-1 inhibitor/antagonist (here IL-1 receptor antagonist) be employed in combination with antioxidants, like acetylcystein, or antiexcitotoxic, like Riluzole, Dexotromethorpan, Lamotrigine, Gabapentin, Topiramate, Nimodipine or Verapamil and the like. Also trophic factors (e.g. BDNF, IGF-1, CNTF) may be employed in such co-therapy approaches.

Before the present invention is described in detail by reference to the appended figures and figure legends and results, it is to be understood that this invention is not limited to the particular methodology, protocols, cells, animal models, reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

The Figures Show:

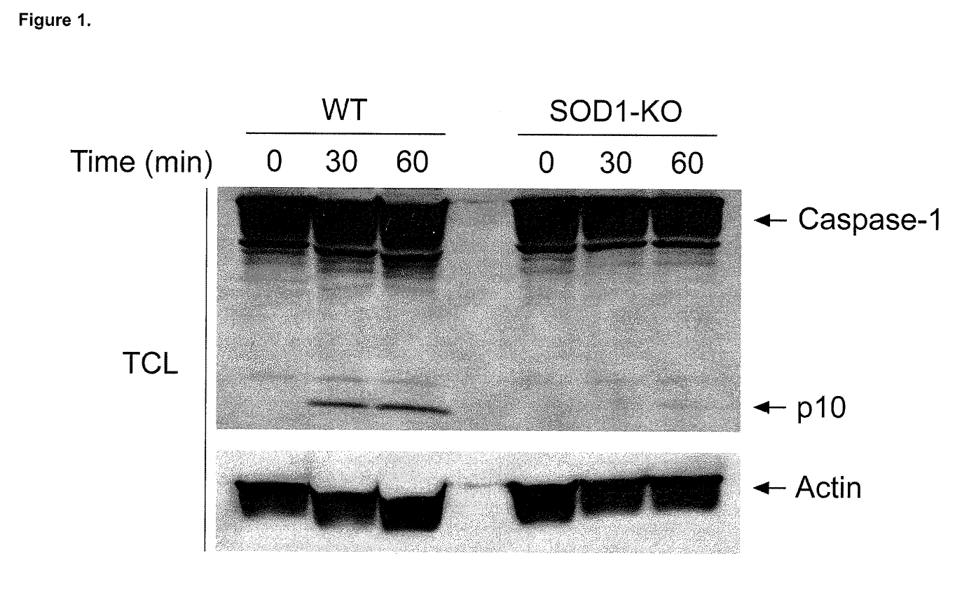

### FIG. 1

### SOD1 Deficiency Impairs Caspase 1 Activation

Peritoneal wild-type and sod1 null (SOD1-KO) macrophages were primed with 500 ng/ml LPS for 3 h and then pulsed with 2 mM ATP. Cell lysates were immunoblotted with an antibody against the p10 subunit of caspase-1. In the sod1 null (SOD1-KO) macrophages caspase-1 activation is impaired as the active subunit p10 cannot be detected after 30 min of activation. The upcoming band after 60 min is much weaker compared to the wild-type.

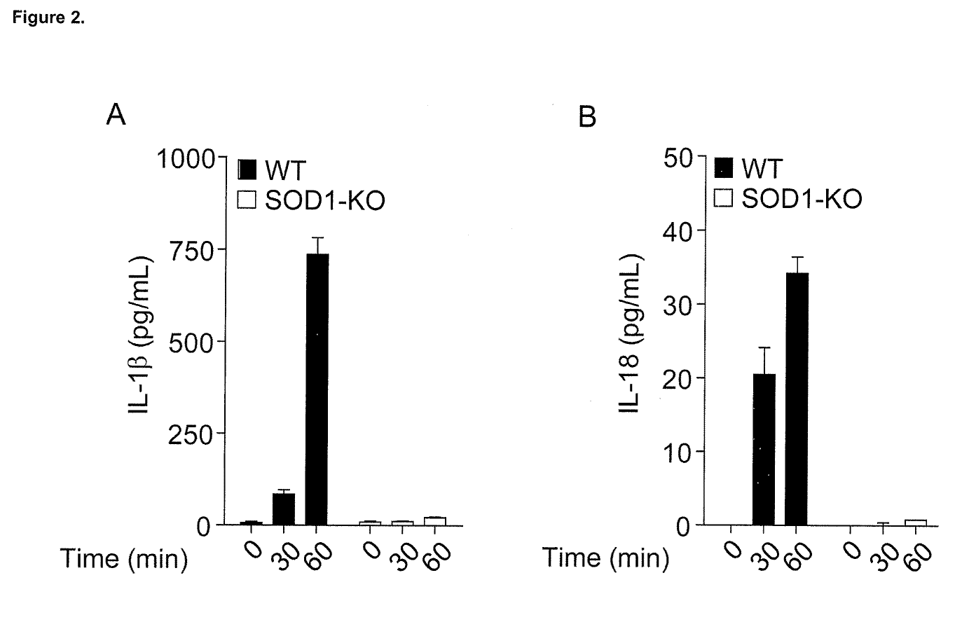

### FIG. 2

### SOD1 Deficiency Impairs Maturation of Il-1 Beta (Il-1β) and Il-18

(A) Peritoneal wild-type and sod1 null (SOD1-KO) macrophages were primed with 500 ng/mL LPS for 3 h and then pulsed with 2 mM ATP. Secretion of mature IL-1 beta (IL-1β). (A) into the cell supernatant was determined by ELISA. (B) Peritoneal wild-type and SOD1-KO macrophages were stimulated with 2 mM ATP. Secretion of mature IL-18 into the cell supernatant was determined by ELISA. Bars represent the mean±the standard error of the mean (s.e.m.). Secretion of IL-1 beta (IL-1β) and IL-18 is strongly impaired in sod1 null (SOD1-KO) macrophages compared to WT. This is due to the impaired cleavage of caspase-1 demonstrated in FIG. 1.

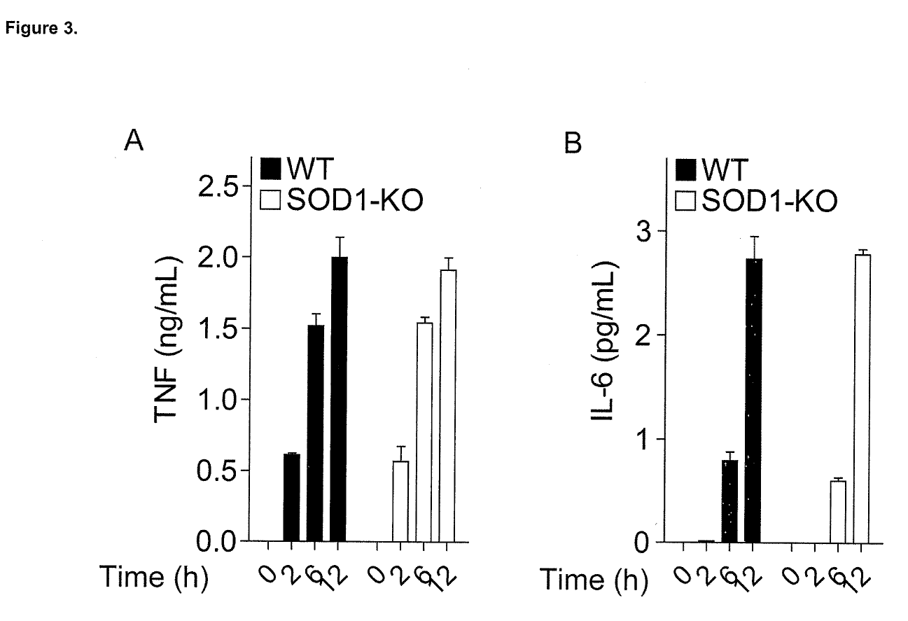

### FIG. 3

### Secretion of Caspase-1 Independent Cytokines is Not Affected in SOD1 Null (SOD1-KO) Macrophages

(A, B) Peritoneal macrophages were cultured in the presence 500 ng/ml LPS. Secretion of the caspase-1 independent cytokines tumor necrosis factor (TNF)(A) and IL-6 (B) into the supernatant was determined by ELISA. The secretion of both TNF and IL-6 is not affected in sod1 null (SOD1-KO) macrophages. Bars represent the mean±s.e.m. This shows that the effect on IL-1beta (IL-1β) and IL-18 demonstrated in FIG. 2 is specific for caspase-1 dependent cytokines.

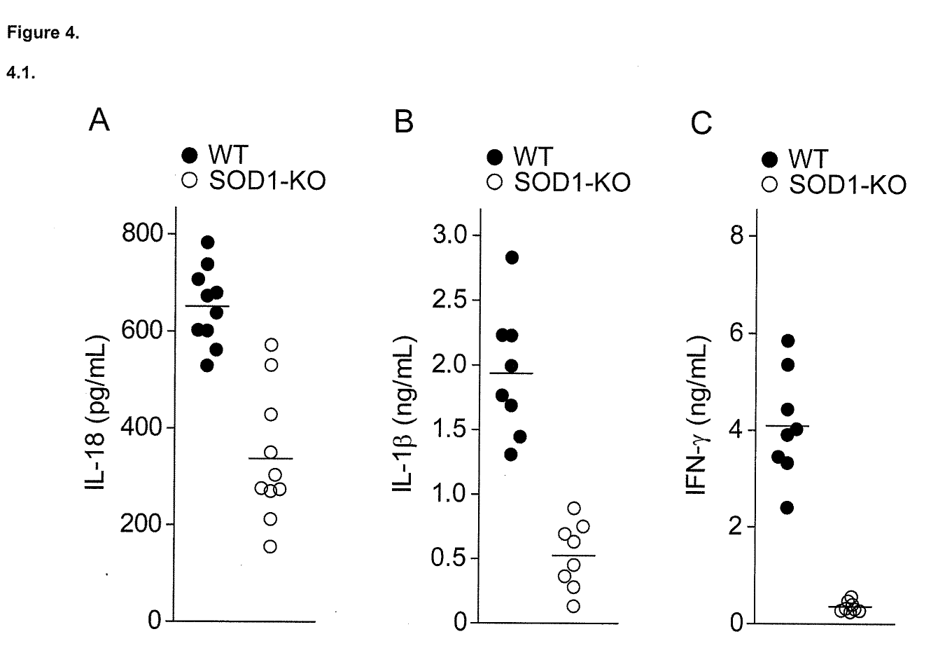

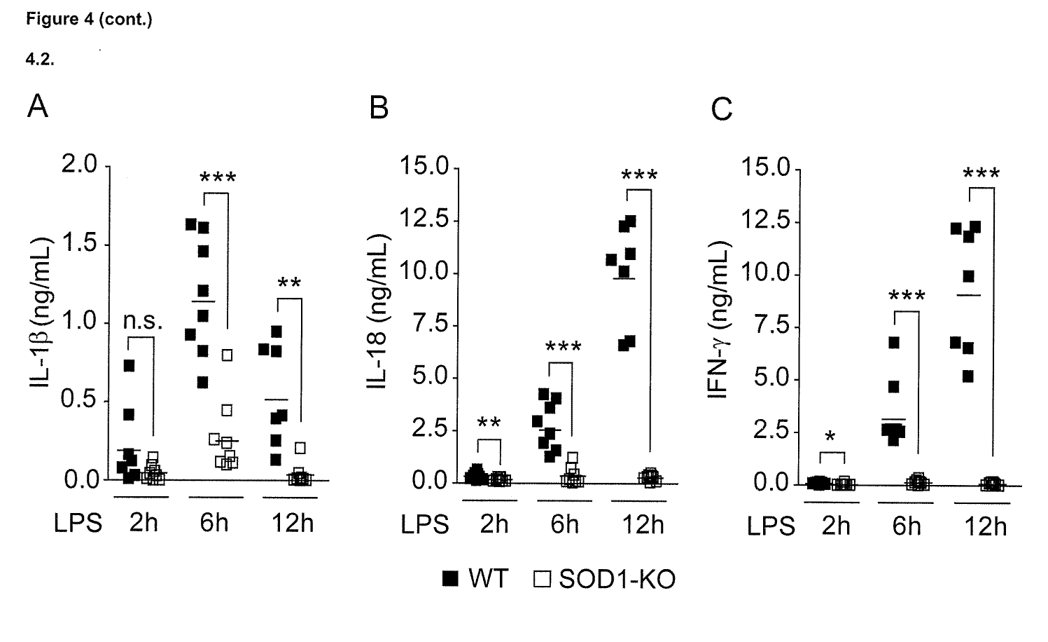

### FIG. 4

### SOD1 Null (SOD1-KO) Mice Show a Reduced Production of Caspase-1 Dependent Cytokines

### (FIGS. 4.1 A, B, C)

Age-Matched Female Wild-Type and Sod1 Null (Sod1-KO) Mice were Injected intraperitoneally with E. coli LPS (15 mg/kg). Serum levels of IL-18 (A) IL-1β(B) and IFN-γ (C) were determined at 2 h (A) and 6 h (B, C) after challenge. Lines indicate the mean serum levels. The serum levels of all three cytokines were substantially reduced in SOD1-KO mice compared to wild-type controls. These results are consistent with the previously presented data from peritoneal macrophages (FIG. 2). Secretion of caspase-1 dependent cytokines IL-1 beta (IL-1β) and IL-18 is reduced due to impaired activation of caspase-1 (FIG. 1). IFN-gamma (IFN-γ) secretion is reduced as it depends on the presence of IL-18 which is an IFN-gamma inducing factor.

(FIGS. 4.2 A, B, C) Age matched female wild-type and SOD1-KO mice were injected intraperitoneally with E. coli LPS (15 mg/kg). Serum levels of IL-1 b (A), IL-18 (B) and IFN-g (C) were determined at 2, 6 and 12 hours after challenge. Lines indicate the mean serum levels. The serum levels of all three cytokines were significantly reduced in SOD1-KO mice compared to wild-type controls. These results are consistent with the previously presented data from peritoneal macrophages (FIG. 2). Secretion of Caspase-1 dependent cytokines IL-1b and IL-18 is reduced due to impaired activation of caspase-1 (FIG. 1). IFN-g secretion is reduced as it depends on the presence of IL-18 which is an IFN-g inducing factor. (*, P<0.01; **, P<0.001; ***, P<0.0001; NS, not significant.)

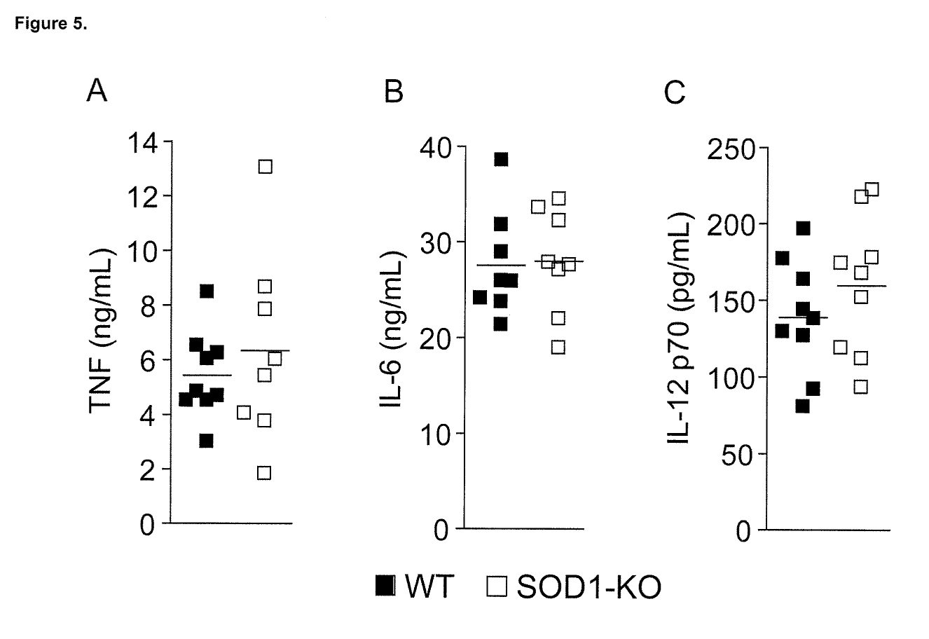

### FIG. 5

### Production of Caspase-1 Independent Cytokines is Not Affected iN SOD1 Null Mice

(A, B, C) Age-matched female wild-type, sod1 null (SOD1-KO) mice were injected intraperitoneally with E. coli LPS (15 mg/kg). Serum levels of TNF (A), IL-6 (B) and IL-12p70 (C) were determined 2 h, (A, C) or 6 h (B) after challenge. Lines indicate the mean serum levels. There is no difference in the secretion of these caspase-1 independent cytokines in sod1 null (SOD1-KO) mice compared to wild-type controls. This demonstrates the specific reduction of the secretion of caspase-1 dependent cytokines in SOD1-KO mice (FIG. 4).

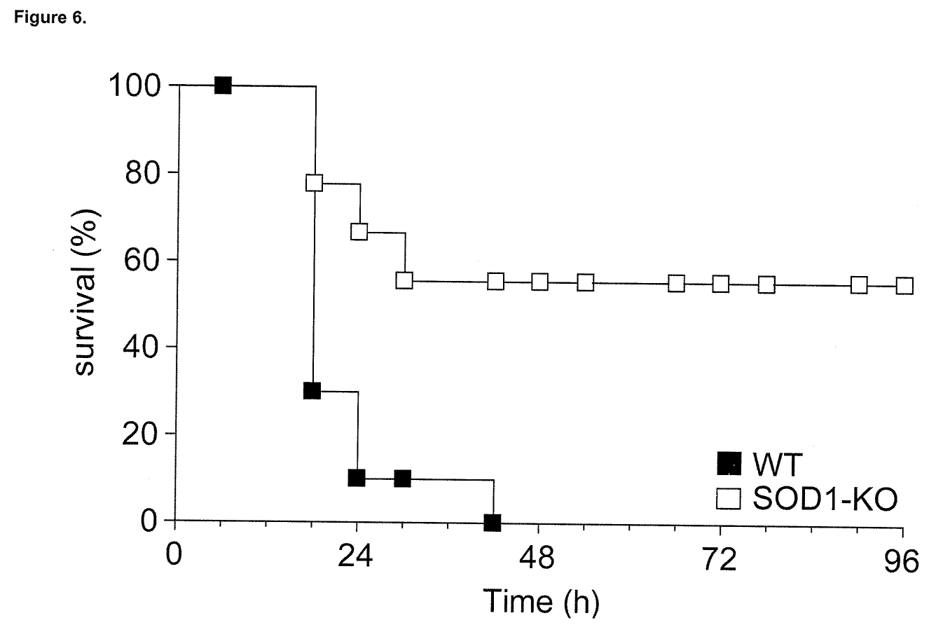

### FIG. 6

### SOD1 Null Mice are More Resistant to Lps-Induced Septic Shock.

Age-matched female wild-type and sod1 null (SOD1-KO) mice were injected intraperitoneally with E. coli LPS (15 mg/kg). Survival of wild-type (n=10) and sod1 null (SOD1-KO) (n=9) mice was monitored. SOD1 null (SOD1-KO) mice are significantly more resistant to LPS-induced septic shock compared to wild-type controls (P=0,0041; Log rank test). These data indicate that inhibition of caspase-1 or caspase-1 dependent cytokines is a potential therapy for septic shock.

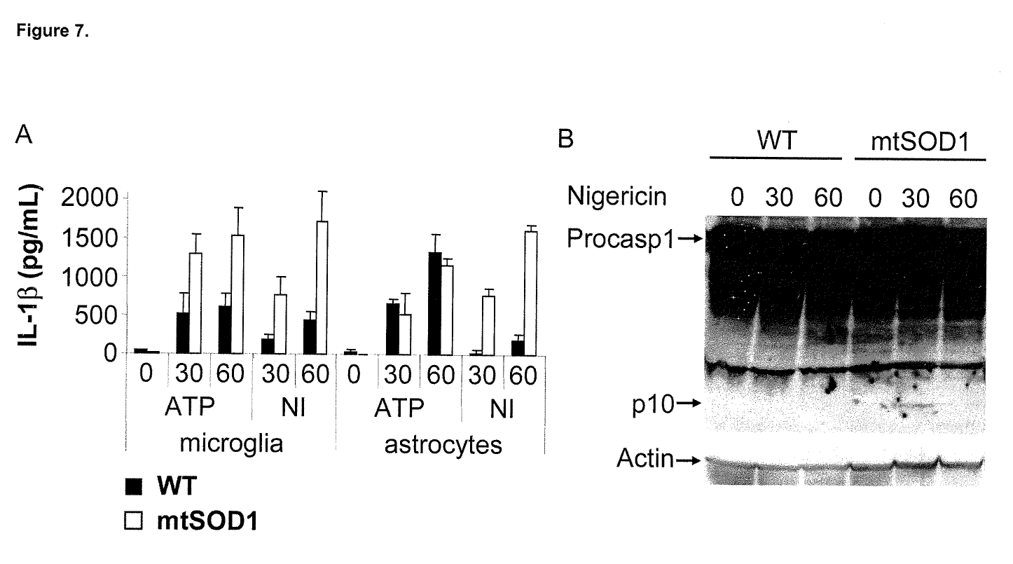

### FIG. 7

Secretion of IL-1beta (IL-1β) and Activation of Caspase-1 are Increased in Microglia and Astrocytes from Mutant Human G93A-SOD1 Transgenic Mice

Microglia and astrocytes were isolated from new born (neonatal) mouse brains of wild type mice and mutant human G93A SOD1 transgenic mice (mtSod1). (A) Microglia and astrocytes were primed with 500 ng/mL LPS for 3 h and then pulsed with 2 mM ATP or 5 μM nigericin, respectively. Secretion of mature IL-1 beta (IL-1β) into the cell supernatant was determined by ELISA at 30 and 60 min after stimulation. Bars represent the mean±s.e.m. Microglia from mtSOD1 mice show an increased secretion of IL-1beta (IL-1β) after stimulation with ATP or nigericin compared to wild type controls. Astrocytes from wild-type and mtSOD1 animals show no difference in IL-1 beta (IL-1β) secretion when stimulated with ATP. In contrast, mtSOD1 astrocytes secrete much more IL-1 beta (IL-1β) than wild-type astrocytes after stimulation with nigericin.

(B) Wild-type and mtSOD1 astrocytes were primed with 500 ng/mL LPS for 3 h and then pulsed with 5 μM nigericin for 30 or 60 min. Cell lysates were immunoblotted with an antibody against the p10 subunit of caspase-1. The active subunit of caspase-1, p10, can only be detected in mtSOD1 astrocytes after stimulation with nigericin.

Taken together these experiments demonstrate that astrocytes and microglia from mtSOD1 mice are hyperreactive to caspase-1 stimuli compared to wild-type control cells.

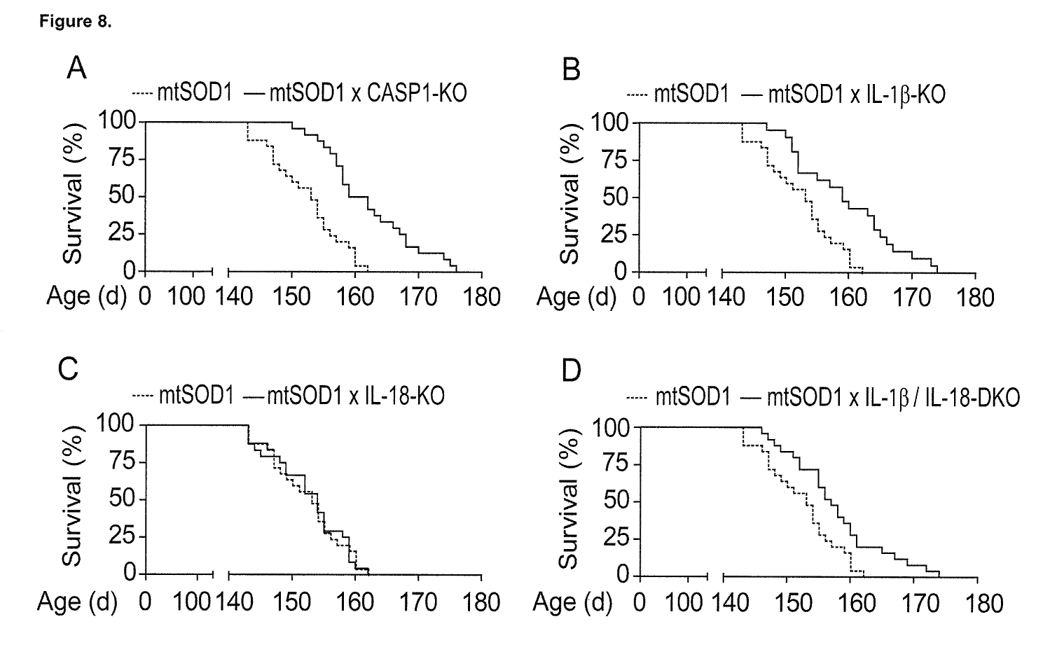

### FIG. 8

### IL-1βContributes to ALS Pathogenesis

(A-D) MtSOD1 transgenic mice were crossed with CASP1-deficient (n=24) (A), IL-1β-deficient (n=21) (B), IL-18-deficient (n=24) (C) and IL-1β/IL-18-double-deficient (n=25) (D) mice and survival of the offspring was monitored and compared with mtSOD1 mice (n=25). (A) MtSOD1×CASP-1-KO animals live significantly longer than mtSOD1 animals (median survival mtSOD1×CASP1-KO=162 days; median survival mtSOD1=153 days; P<0.0001; Log rank test). This demonstrates that caspase-1 contributes to the pathogenesis of ALS.(B) MtSOD1×IL-1β-KO animals live significantly longer than mtSOD1 animals (median survival mtSOD1×IL-1β-kO=159 days; median survival mtSOD1=153 days; P=0.0006; Log rank test). This demonstrates that IL-1β contributes to the pathogenic processes in ALS. (C) MtSOD1×IL-18-KO animals do not live longer than mtSOD1 mice (median survival mtSOD1×Il-18-KO=154 days; median survival mtSOD1=153 days; P=0.9265; Log rank test). This indicates that IL-1βdoes not contribute to ALS pathogenesis. (D) MtSOD1×IL-1β/IL-18-DKO animals live significantly longer than mtSOD1 mice (median survival mtSOD1×IL-1β/IL-18-DKO=157 days; median survival mtSOD1=153 days; P=0.0061; Log rank test). These data demonstrate that caspase-1 affects ALS pathogenesis and is mediated by the caspase-1 dependent cytokine IL-113.

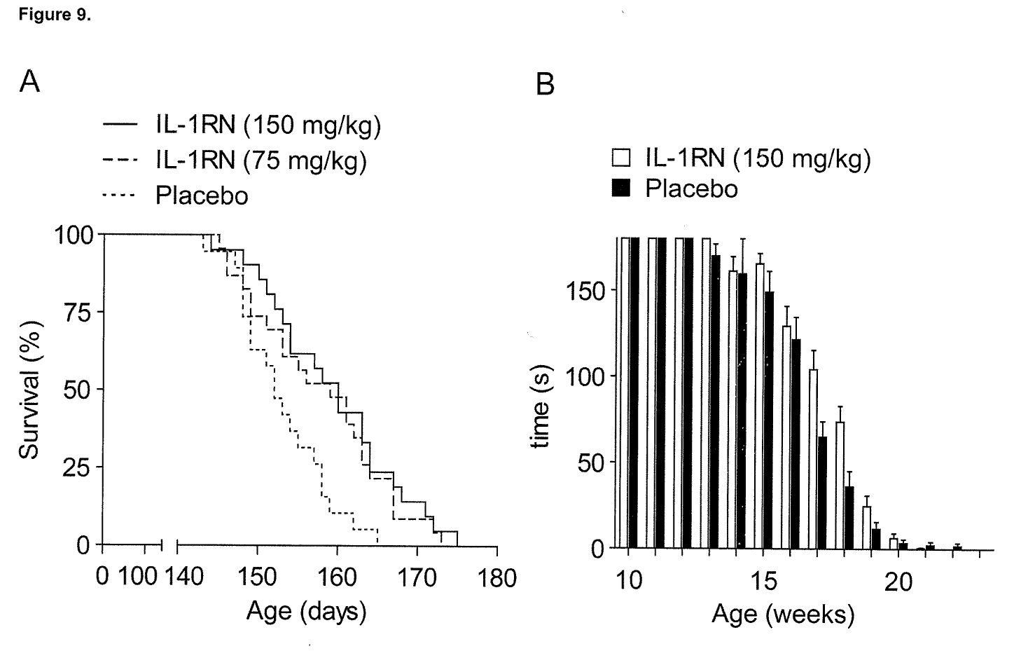

### FIG. 9

Treatment of Mutant SOD1 Transgenic Mice with IL-1RN Increases the Median Survivial of mtSOD1 Mice and Mitigates Disease Progression

(A, B) Starting with the age of 70 days, mtSOD1 mice were injected intraperiotneally daily with either 150 mg/kg of IL-1RN (Kineret®/Anakinra) (n=21), 75 mg/kg IL-1RN (n=23) or with placebo (with the carrier alone) (n=19). The treatment continued until the death of the animal. (A) Survival of the animals was monitored. IL-1RN treated animals lived significantly longer than placebo treated controls (median survival placebo=152 days; 150 mg/kg IL-1RN=160 days, (P=0.0036; Log rank test); 75 mg/kg Il1=159 days (P=0.0145; Log rank test)). (B) Once a week motor neuron performance of each mouse was analysed using the hanging wire test. Bars represent the mean±s.e.m. Treatment with IL-1RN slows down disease progression and improves the motor performance significantly in week 17 (P=0.0123; two-tailed Student's t-test) and in week 18 (P=0.0095; two-tailed Student's t-test) as assessed by the hanging wire test. These results demonstrate that IL-1RN treatment slows down ALS disease progression in mtSOD1 mice. Disease onset is not affected by the treatment (B) whereas survival is prolonged (A) and disease symptoms were ameliorated (B).

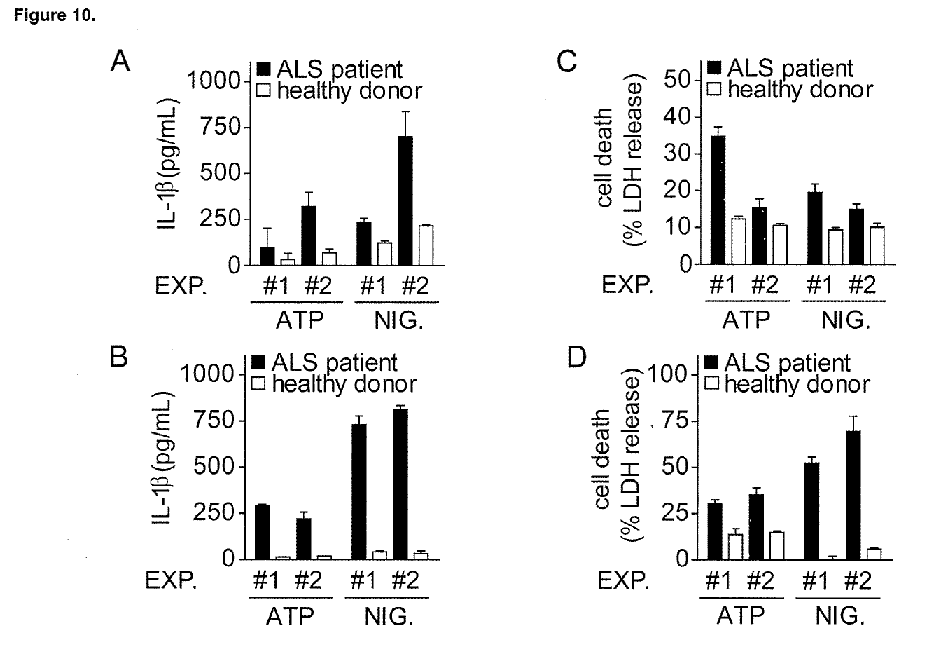

### FIG. 10

Monocytes and Macrophages from ALS Patients are Hyperreactive to Caspase-1 Stimulation

Peripheral blood monocytes (A, C) and macrophages (B, D) were isolated from the blood of two ALS patients with mutations in the SOD1 gene and from two healthy controls. The cells were primed by LPS (500 ng/ml) for 3 h and then stimulated with caspase-1 activating agents (ATP, nigericin). IL-1 beta (IL-1β) secretion was determined by ELISA after 60 min (A, B) and cell death was assessed by LDH release after 120 min (C, D). Both monocytes and macrophages of ALS patients show higher levels of mature IL-1 beta (IL-1β) and LDH in the cell supernatant than healthy controls indicating hyper responsiveness to caspase-1 stimulation.

1. A method for treating, preventing or ameliorating a chronic neurodegenerative disorder, said method comprising administering to a subject in need of such a treatment, prevention or amelioration a specific inhibitor of a caspase I-dependent cytokine.

2. A specific inhibitor of a caspase I-dependent cytokine for treating, preventing or ameliorating a neurodegenerative disorder.

3. (canceled)

4. The method of claim 1, wherein said caspase I-dependent cytokine is selected from the group consisting of interleukin-1 (IL-1), interleukin-18 (IL-18) interleukin-33 (IL-33) and interferon-gamma (IFN-γ).

5. The method of claim 4, wherein said interleukin-1 (IL-1) is IL-1α and/or IL-1β.

6. The method of claim 1, wherein said a specific inhibitor of a caspase I-dependent cytokine is selected from the group consisting of neutralizing antibody or an antibody derivative or a fragment thereof to interleukin-1 (IL-1), interleukin-33 (IL-33), interleukin-18 (IL-18) and/or interferon-gamma (IFN-γ), antisense oligonucleotides specifically interacting with nucleic acid molecules encoding interleukin-1 (IL-1), interleukin-18 (IL-18), interleukin-33 (IL-33) or interferon-gamma (IFN-γ), siRNA or RNAi directed against interleukin-1 (IL-1), interleukin-18 (IL-18) or interferon-gamma (IFN-γ), ribozymes specifically interacting with nucleic acid molecules encoding for functional interleukin-1 (IL-1), interleukin-33 (IL-33), interleukin-18 (IL-18) or interferon-gamma (IFN-γ), an interleukin-1 (IL-1) receptor antagonist, a interleukin-18 (IL-18) receptor antagonist and a interferon-gamma (IFN-γ) receptor antagonist.

7. The method of claim 6, wherein said interleukin-18 (IL-18) antagonist is interleukin-18 binding protein (Tadakinig-alpha).

8. The method of claim 6, wherein said interferon γ (IFN-γ) antagonist is soluble IFN-gamma receptor.

9. The method of claim 6, wherein said interleukin-1 (IL-1) antagonist is selected from the group consisting of anakinra, IL-1 TRAP and CDP 484.

10. The method of claim 6, wherein said receptor antagonist is a proteineous or peptide antagonist.

11. The method of claim 10, wherein said receptor antagonist is an interleukin-1 receptor antagonist.

12. The method of claim 11, wherein said receptor antagonist is a human interleukin-1 receptor antagonist.

13. The method of claim 11, wherein said human interleukin-1 receptor antagonist is anakinra (Kineret®).

14. The method of claim 1, wherein said neurodegenerative disorder is selected from the group consisting of lower motor neuron disease, amyotrophic lateral sclerosis (ALS), Alzheimer's disease, Parkinson's disease, and Huntington's disease,

15. The method of claim 14, wherein said amyotrophic lateral sclerosis (ALS) is familial amyotrophic lateral sclerosis (FALS).

16. The method of claim 15, wherein said familial amyotrophic lateral sclerosis (FALS) is linked to a mutation/variant of the Cu/Zn superoxide dismutase (SOD1).

17. The method of claim 14, wherein said lower motor neuron disease is selected from the group consisting of progressive muscular atrophy (PMA), spinal muscular atrophy (SMA) and Kennedy's disease.
